(19) United States
(12) Patent Application Publication
(10) Pub. No.: US 2010/0094062 A1
(43) Pub. Date: Apr. 15, 2010

(54) CETANE NUMBER INCREASING PROCESS AND ADDITIVE FOR DIESEL FUEL

(76) Inventors: Carlos René Klotz Rabello, Rio de Janeiro (BR); Yuri Kokitsu Ferreira, Niteroi (BR); Raphael Bezerra de Menezes, Rio de Janciro (BR)

Correspondence Address: NIXON & VANDERHYE, PC, 901 NORTH GLEBE ROAD, 11TH FLOOR, ARLINGTON, VA, 22203, US

(21) Appl. No.: 12/596,016

(22) PCT Filed: Apr. 18, 2007
(86) PCT No.: PCT/GB07/01424

§ 371 Date: Dec. 4, 2009

(51) Int. Cl. C07C 43/00

(52) U.S. Cl. 568/589

(57) ABSTRACT

A cetane number increasing process and additive for diesel fuel, this additive being obtained by means of a less complex and more economic process which seeks to use by products in excess of supply on the market and to optimize the installed capacity of existing plants, the process producing a mixture of nitrated glycerol diethers is represented by the following general formula (I) where R can be: a hydrogen atom; or an R′—O group; and where R′ can be an alkene or alkyne or an unsaturated hydrocarbon formed by a number of carbon atoms ranging from 4 to 10 carbons.

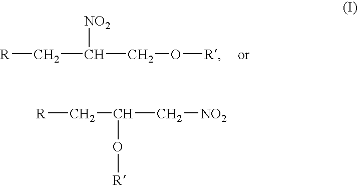

### FIELD OF THE INVENTION

The field of application of the present invention is cetane number increasing processes and additives for diesel fuel. More particularly additives produced from glycerine and alkenes and/or alkynes of four or more carbon atoms, used to increase the cetane number in a diesel fuel.

### STATE OF THE ART

The constant development of the petrochemical industry is making obsolete processes and products hitherto considered to be strategic and of fundamental importance. In some cases, the new processes generate an abundance of by-products. The use of these by-products, together with the use of obsolete plants has been a considerable challenge for the industry, which is searching for the possibility of creating processes which are technically simple and economically viable.

According to U.S. Pat. No. 6,015,440, a large volume of compounds such as methyl tert-butyl ether (MTBE) and ethyl tert-butyl ether (ETBE) has be used as additives for petrol. However, environmental and legal restrictions have caused drastic reductions in these compounds, and resulted in a decrease in the scale of production plants, and consequently the obsolescence of the process.

Environmental and legal restrictions have also driven the development of biodiesel. One of the by-products of the process for obtaining biodiesel is glycerine. In general, glycerine is produced in a quantity of approximately 10% by weight of the quantity of biodiesel produced. Thus, for a forecast future growth of production of biodiesel to a thousand million gallons (approximately 3,785,412 m³), 400,000 tonnes of glycerine would be produced. As things stand, the market is incapable of absorbing this substantial increase in supply. This being the case, the tendency in the short term is for glycerine to become an expensive industrial waste product which is difficult to handle.

As regards diesel-based fuels, the quality of ignition in compression ignition engines can be measured by means of the cetane number.

Various chemical compounds are known to function as cetane number increasing additives in diesel fuels. In Brazil, the principal additive employed is 2-ethylhexyl nitrate, produced from 2-ethylhexanol. To date 2EHN is not produced in Brazil, so that the petrochemical industry is obliged to import the compound employed and to submit to market conditions. Currently, given the constant increases in the price of propene, fundamental starting material in the petrochemical industry, and in the process for obtaining 2-ethylhexanol, the costs of importing 2EHN are on the rise. At the same time, attempts to produce 2EHN domestically have not proved to be economically viable.

Various patents protect cetane number increasing additives for diesel fuel. U.S. Pat. No. 4,549,883 protects some dinitrated esters as additives for improving the cetane number.

U.S. Pat. No. 4,473,378 protects a cetane number increasing additive formed by a mixture of equal quantities of 2-methyl-2-nitropropyl nitrate and isooctyl nitrate. U.S. Pat. No. 4,536,190 protects a mixture of equal quantities of 2-methyl-2-nitropropyl nitrate and 5,6-cyclopentene-2-norbonyl nitrate as a cetane number increasing additive.

European patent EP 0 903 395 protects diesel fuel compositions containing dimethoxyethane (DMET) and/or dimethoxypropane (DMPP) as cetane number increasing additives. Other dialkoxyalkanes can be added to said composition.

Thus, the tendency in the state of the art is towards a less complex and economically viable process for producing a cetane number increasing additive for diesel fuel. The process is preferably capable of being carried out with economies of scale by making rational use of the installed capacity of industrial plants and of by-products in excess supply on the market.

### SUMMARY OF THE INVENTION

The cetane number increasing process and additive for diesel fuel, the additive being obtained by means of a less complex and more economic process, also seek to use by-products in excess supply on the market, and to optimize the installed capacity of existing plants, by means of the steps described below.
reaction of an alkene or alkyne of four or more carbon atoms with glycerine which may or may not come from biodiesel production plants, using catalysts which can be selected from: conventional catalysts for obtaining MTBE, such as, for example, acidic ion-exchange resins, and catalysts with active sites, such as zeolites;
isolation of the alcohol diethers thus formed, and nitration of the resulting glycerol alcohol diethers, which thereby have the hydroxyl group substituted, forming glycerol diether nitrates.

The aforementioned glycerol diether nitrates confer the intended increase in cetane number on diesel fuel.

### DETAILED DESCRIPTION OF THE INVENTION

The cetane number increasing process and additive for diesel fuel which are the objects of the present invention will be described in detail, identifying the respective components and steps of the production process.

In order to better understand the object of the invention, diesel fuel can be considered to be a mixture of hydrocarbons at the distillation temperature of diesel, which is in the range from 160° C. to 370° C. Within the traditional IUPAC nomenclature, glycerine is known as propanetriol.

The present invention uses glycerine as starting material for synthesizing ethers, which subsequently undergo a nitration process. The composition obtain is then used as a cetane number increasing additive for diesel fuel. Thus, the synthesis of the nitrated glycerol ethers comprises the following steps:
etherification of glycerine with one or more alkenes or alkynes of four or more carbon atoms, and preferably isobutene from the C4 hydrocarbon fraction from a refinery or petrochemical plant. The reaction takes place at a temperature in the range from 50° C. to 150° C. During this stage, catalysts can be used, which can be selected from: conventional catalysts for obtaining MTBE, such as, for example, acidic ion-exchange resins, and catalysts with active sites, such as zeolites. The glycerine is preferably added in a stoichiometric proportion relative to the hydrocarbon. This reaction has an equilibrium, so that the reactants do not undergo complete conversion. Glycerol mono- and diethers are obtained in approximately equal quantities, with a small fraction (close to 2%) of the glycerol triether. This is due to the fact that, as the unsaturated groups are added to the glycerol molecule, it becomes more difficult to add a third group due to steric hindrance. Thus, the monoethers are formed in the greatest quantity, followed by the diethers and finally the glycerol triether.
isolation of the glycerol diethers formed. The isolation step comprises:
   washing the etherified product with water in order to remove unreacted glycerol. Because the glycerol monoethers are soluble in water, they are also removed at the same time, leaving a mixture formed of glycerol diethers (preferably 1,2-di-butoxypropanol and 1,3-dibutoxy-2-propanol) and a small quantity of glycerol triethers. Alternatively, the triether can be separated from the glycerol diethers by means of a process of extraction with a nonpolar organic solvent. This solvent needs to have a low boiling point which differs from the glycerol triether and diethers. In this case, the stream of glycerol and the monoethers is, recycled directly to the process, while the extract containing the glycerol triether and diethers are isolated from the solvent by distillation. The recovered solvent is returned to the process. This alternative is suitable since it consumes less energy than a conventional distillation stage.
   the aqueous phase containing glycerine and the monoethers is sent for distillation. The bottom fraction is composed of glycerine and monoethers, while the top fraction is the water, which is reused in the washing step; and
nitration of the resulting mixture of glycerol diethers and triether.
   the nitration occurs at a temperature of 5° C., using a mixture of sulphuric and nitric acids (HNO₃/H₂SO₄, oleum type) in the proportion 1:1. The residence time in the reactor is 10 seconds;
   under milder conditions, such as temperature below 5° C. and a residence time of approximately one second, a mixture of nitrated glycerol diethers (preferably 1,2-di-butoxypropyl nitrate and 1,3-dibutoxypropyl nitrate). Alternatively, use of a mixture of acetic anhydride and nitric acid in the proportion 1:1 by volume also produces the aforementioned mixture of nitrated glycerol ethers, while acetic anhydride is less acid in character than sulphuric acid;
   under more severe operating conditions, such as, for example, a temperature higher than 500° C. and a sulphuric acid/nitric acid mixture in a proportion of 2:1, a mixture of nitrated glycerol monoether (preferably 1-butoxypropane) and nitrated glycerol diether may be obtained, since under severe reaction conditions one of the ether groups of the diether may be cleaved. Irrespective of which is used, the three compositions obtained can be applied as cetane number increasing additives for diesel fuel.

By this means, a mixture of nitrated glycerol monoether and nitrated glycerol diethers is obtained which are represented by the following general formula:

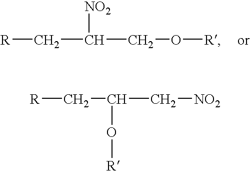

where R can be:
   a hydrogen atom; or
   an R′—O group; and
where R' can be an alkene or alkyne or an unsaturated hydrocarbon formed by a number of carbon atoms ranging from 4 to 10 carbons.

### Example

A sample of diesel fuel from a refinery was mixed with 500 ppm (by volume) of 2-ethylhexyl nitrate (2EHN). Another sample of the same diesel fuel was mixed with 500 ppm (by volume) of 1-butoxypropyl nitrate (1BPN).

The cetane numbers of the samples above and of a sample of the neat diesel fuel were determined by the method of ASTM D6890, entitled “Ignition Quality Test” (IQT). The results are presented in Table 1.

TABLE 1

| Sample | Cetane number |
|---|---|
| Neat diesel fuel | 43.9 |
| Diesel fuel + 500 ppm 2EHN | 49.6 |
| Diesel fuel + 500 ppm 1BPN | 47.3 |

The results found with 1BPN were better than for the neat diesel fuel and quite close to those found with 2EHN. Even so, considerable progress has been made as regards the use of a composition obtained by a simple and economic process.

The description so far of the cetane number increasing process and additive for diesel fuel which are the objects of the present invention should be considered to be of only one possible or several possible embodiments and any specific characteristics introduced therein should be understood only as having been written in order to aid understanding. They should, therefore, not be regarded as in any way defining the invention, which is defined by the scope of the claims below.

1.-25. (canceled)

26. Cetane number increasing additive for diesel fuel obtained by means of a process which comprises the following steps:
etherification reaction between one or more alkenes or alkynes or unsaturated hydrocarbons and glycerine;
isolation of the glycerol or other alcohol diethers formed; 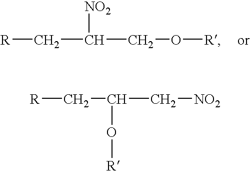
nitration of the resulting glycerol alcohol diethers, which thus have the hydroxyl group substituted, to form glycerol monoether or diether nitrates, characterized (i) by the use of glycerine from plants for producing biodiesel and/or (2) in that the additive obtained has the following general formula:
where R can be:
a hydrogen atom; or
an R-0 group; and
where R' can be an alkene or alkyne or an unsaturated hydrocarbon formed by a number of carbon atoms ranging from 4 to 10 carbons.

27. Cetane number increasing additive for diesel fuel according to claim 26, characterized in that the additive is obtained in a processing plant for methyl tert-butyl ether.

28. Cetane number increasing additive for diesel fuel according to claim 26, characterized in that the unsaturated hydrocarbon comprises isobutene.

29. Cetane number increasing additive for diesel fuel according to claim 26, characterized by the use of glycerine from plants for producing biodiesel.

30. Cetane number increasing additive for diesel fuel according to claim 26, characterized in that the etherification reaction is catalysed by means of catalysts which can be selected from: conventional catalysts for obtaining methyl tert-butyl ether, and/or catalysts with acid active sites.

31. Cetane number increasing additive for diesel fuel according to claim 30, characterized in that the etherification reaction is catalysed by means of catalysts which can be selected from: zeolites and/or acidic ion-exchange resins.

32. Cetane number increasing additive for diesel fuel according to claim 26, characterized in that water is used to remove the etherified product in the step of separating the etherified products.

33. Cetane number increasing additive for diesel fuel according to claim 26, characterized in that the step of isolating the etherified product is carried out by means of extraction with one or more nonpolar organic solvent(s).

34. Cetane number increasing additive for diesel fuel according to claim 26, characterized in that during the step of separating the alcohol diethers formed a mixture formed by 1,2-dibutoxypropanol and 1,3-dibutoxy-2-propanol and a quantity, preferably a small quantity, of glycerol triethers is left.

35. Cetane number increasing additive for diesel fuel according to claim 26, characterized in that the step of nitrating the diethers obtained occurs in a temperature range from 0° C. to 10° C. in the presence of an oleum type mixture of nitric acid and sulphuric acid in the proportion of about 1:1, with a residence time in a reactor in the range from 1 to 10 seconds.

36. Cetane number increasing additive for diesel fuel according to claim 26, characterized in that the nitration step is performed in the presence of an oleum type mixture of nitric acid and sulphuric acid in the proportion of about 2:1.

37. Cetane number increasing additive for diesel fuel according to claim 26, characterized in that the nitration step is performed in the presence of a mixture of acetic anhydride and nitric acid in the proportion of about 1:1.

38. Cetane number increasing additive for diesel fuel according to claim 26, characterized in that during the step of nitrating the glycerol diethers obtained a mixture of 1,2-dibutoxypropane nitrate and 1,3-dibutoxypropane nitrate or a mixture of 1-butoxypropyl nitrate 1,2-dibutoxypropane nitrate and 1,3-dibutoxypropane nitrate is formed.

39. Process for obtaining a cetane number increasing additive for diesel fuel, which includes the following steps:
etherification reaction between one or more alkenes or alkynes or unsaturated hydrocarbons and glycerine;
isolation of the glycerol or other alcohol diethers thus formed, and
nitration of the resulting glycerol alcohol diethers, which thus have the hydroxyl group substituted, forming glycerol monoether or diether nitrates, characterized (1) by the use of glycerine from plants for producing biodiesel and/or (2) in that the additive obtained has the following general formula: 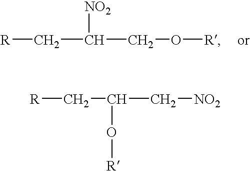
where R can be:
a hydrogen atom; or
an R′—O group; and
where R′ can be an alkene or alkyne or an unsaturated hydrocarbon formed by a number of carbon atoms ranging from 4 to 10 carbons.

40. Process for producing a cetane number increasing additive for diesel fuel according to claim 39, characterized in that the additive is obtained in a processing plant for methyl tert-butyl ether.

41. Process for obtaining a octane number increasing additive for diesel fuel according to claim 39, characterized in that the unsaturated hydrocarbon comprises isobutene.

42. Process for obtaining a cetane number increasing additive for diesel fuel according to claim 39, characterized in that the esterification reaction is catalysed by means of catalysts which can be selected from: conventional catalysts for obtaining methyl tert-butyl ether, and/or catalysts with acid active sites.

43. Process for obtaining a cetane number increasing additive for diesel fuel according to claim 42, characterized in that the esterification reaction is catalysed by means of catalysts which can be selected from: zeolites and/or acidic ion-exchange resins.

44. Process for obtaining a cetane number increasing additive for diesel fuel according to claim 39, characterized in that water is used to remove the etherified product in the step of separating the etherified products.

45. Process for obtaining a cetane number increasing additive for diesel fuel according to claim 39, characterized in that the step of isolating the etherified product is carried out by means of extraction with one or more nonpolar organic solvent(s).

46. Process for obtaining a cetane number increasing additive for diesel fuel according to claim 39, characterized in that during the step of separating the alcohol diethers formed, a mixture formed by 1,2-dibutoxypropanol and 1,3-dibutoxy-2-propanol and a small quantity, preferably a small quantity, of glycerol triethers is left.

47. Process for obtaining a cetane number increasing additive for diesel fuel according to claim 39, characterized in that the step of nitrating the diethers obtained occurs in a temperature range from 0° C. to 10° C. in the presence of an oleum type mixture of nitric acid and sulphuric acid in the proportion of about 1:1, with a residence time in a reactor in the range from to 10 seconds.

48. Process for obtaining a cetane number increasing additive for diesel fuel according to claim 39, characterized in that the nitration step is performed in the presence of an oleum type mixture of nitric acid and sulphuric acid in the proportion of about 2:1.

49. Process for obtaining a cetane number increasing additive for diesel fuel according to claim 39, characterized in that the nitration step is performed in the presence of a mixture of acetic anhydride and nitric acid in the proportion of about 1:1.

50. Process for obtaining a cetane number increasing additive for diesel fuel according to claim 39, characterized in that during the step of nitrating the glycerol diethers obtained a mixture of 1,2-dibutoxypropane nitrate and 1,3-dibutoxypropane nitrate or a mixture of 1-butoxypropyl nitrate 1,2-dibutoxypropane nitrate and 1,3-dibutoxypropane nitrate is formed.
